(19) United States
(12) Patent Application Publication
(10) Pub. No.: US 2004/0133218 A1
(43) Pub. Date: Jul. 8, 2004

(54) Wound clamp

(76) Inventors: Steven T. Charles, Germantown, TN (US); Dyson W. Hickingbotham, Stouchsburg, PA (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/337,532

(22) Filed: Jan. 7, 2003

(51) Int. Cl. A61B 17/08

(52) U.S. Cl. 606/151

(57) ABSTRACT

A wound clamp having two substantially identical halves. The clamp halves are biased together using a spring or elastomeric band. Prongs or needles integrally formed with the halves cause the clamp to be engaged firmly on the conjunctival and scleral tissue about the wound. The biasing of the clamp halves toward each other provide for a tight sealing of the wound.

### BACKGROUND OF THE INVENTION

This invention relates generally to the field of microsurgery and, more particularly, to ophthalmic microsurgery.

During ophthalmic microsurgery, it is often necessary to remove, dissect, cut, delaminate, coagulate or otherwise manipulate delicate tissues within the eye. Microsurgical tools, such as vitreous cutters, infusion cannulas, micro scissors, micro forceps, illuminated laser probes and other devices are generally used. These devices generally are inserted through one or more surgical incisions in the sclera, a sclerotomy, and different tools may be used during different parts of the surgical procedure. The repeated insertion and removal of these instruments can allow vitreous humor and fluids to escape the eye out through the sclerotomy, increasing the potential for softening of the globe, bleeding, traction on the retina and/or introduction of bacteria into the eye as well as increased healing time.

Therefore, a need continues to exist for a simple, inexpensive clamp for holding the sclerotomy tightly closed during surgery.

### BRIEF SUMMARY OF THE INVENTION

The present invention improves upon the prior art by providing a wound clamp having two substantially identical halves. The clamp halves are biased together using a spring or elastomeric band. Prongs or needles integrally formed with the halves cause the clamp to be engaged firmly on the conjunctival and scleral tissue about the wound. The biasing of the clamp halves toward each other provide for a tight sealing of the wound.

Accordingly, one objective of the present invention is to provide a simple, inexpensive clamp for holding the sclerotomy tightly closed during surgery.

Another objective of the present invention is to provide a clamp have two clamp halves that are biased together using a spring or elastomeric band.

Another objective of the present invention is to maintain alignment of the conjunctiva and scleral incisions if trans-conjunctival surgery is performed.

Still another objective of the present invention is to facilitate introduction of the instruments through the surgical wound.

These and other advantages and objectives of the present invention will become apparent from the detailed description and claims that follow.

### BRIEF DESCRIPTION OF THE DRAWINGS

FIG. 1 is a top perspective view of a first embodiment of the wound clamp of the present invention.

FIG. 2 is a bottom perspective view of a first embodiment of the wound clamp of the present invention.

FIG. 3 is a top perspective view of a second embodiment of the wound clamp of the present invention.

FIG. 4 is a bottom perspective view of a second embodiment of the wound clamp of the present invention.

### DETAILED DESCRIPTION OF THE INVENTION

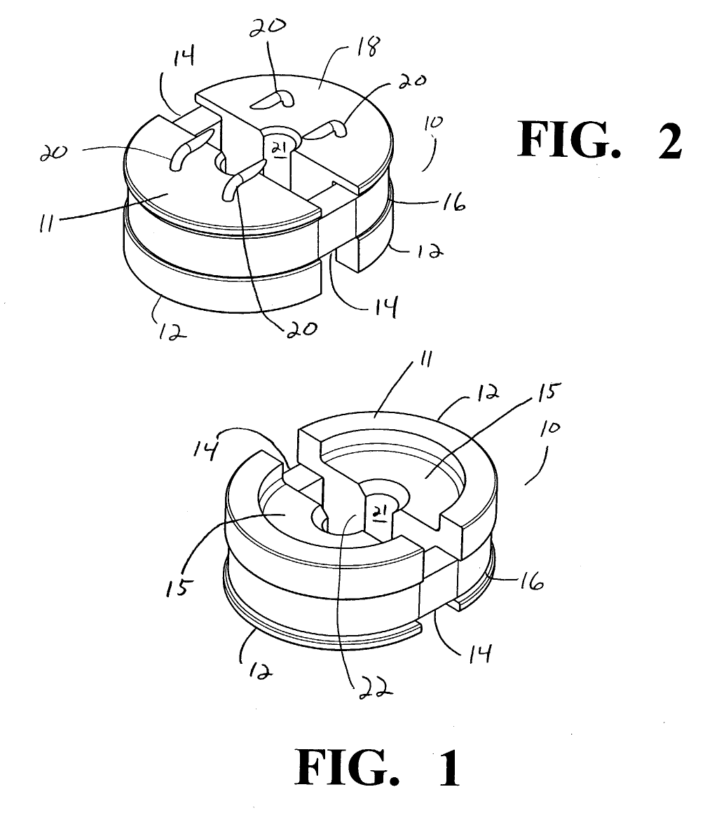

As best seen in FIGS. 1 and 2, in a first embodiment, wound clamp 10 generally consists of body 11 made up of symmetrical halves 12 that are biased together by elastomeric band 14. Band 14 generally fits within groove 16 in halves 12. As best seen in FIG. 2, bottom 18 of halves 12 may contain a plurality of hooks or prongs 20. Halves 12 may be molded or formed from any suitable material, such as thermoplastic, and may contain a lubricious material, such as polytetrafluoroethylene (TEFLON®), and/or a luminous material so as to aid in visualizing clamp 10 in a darkened room. Each of halves 12 contain recess 15 and cut out portions 21 that together define central aperture 22 when halves 12 are biased together. Aperture 22 allows a surgical tool (not shown) to be inserted into a wound when clamp 10 is placed over a surgical site and halves 12 pulled together by band 14. Portions 21 may also be flared, beveled or chamfered to assist in the introduction of the surgical tool into the wound. Recess 15 allows for multiple clamps 10 to nest together during storage, shipping or insertion so as not to damage prongs 20. Band 14 may be made from any suitable elastomeric material, such as silicone rubber. Prongs 20 may be made from any suitable material, such as stainless steel, suture materials such as polypropylene or modified surgical staples and may be integrally formed within halves 12 during the molding process or attached to halves 12 by any suitable process, such as adhesive.

In use, halves 12 of clamp 10 are forced apart by a probe, lance or other suitable tool (not shown) and placed over a surgical incision so that prongs 20 contact the tissue around the incision. The tool is then removed, and the elastomeric nature of band 14 causes halves 12 to be pulled together, thereby forcing prongs 20 in the tissue around the incision and pulling the incision closed and causing clamp 10 to engage firmly on the tissue about the wound. Surgical tools may be inserted into the incision through aperture 22 without causing excessive opening of the incision. In addition, halves 12 may spread apart slightly to enlarge aperture 22 without prongs 20 becoming dislodged from the tissue so as to allow foreign bodies or slightly larger instruments to exit the wound.

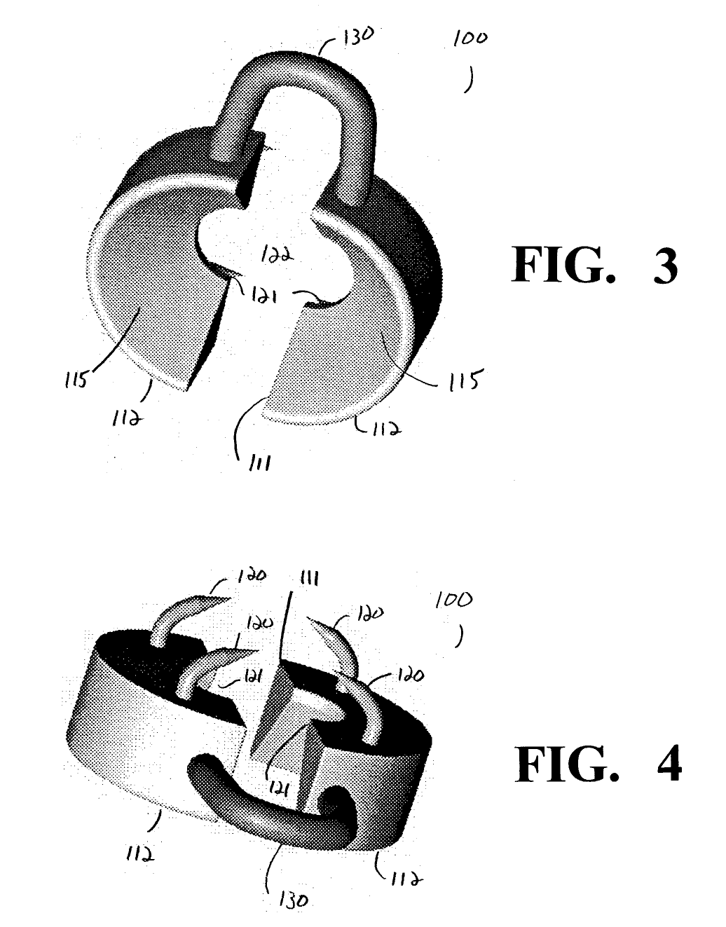

A second embodiment of the present invention, best seen in FIGS. 3 and 4, clamp 100 is of similar construction as clamp 10, body 111 having halves 112, halves 112 having portions 121 that form aperture 122 and prongs 120 and recess 115. Instead of band 14, halves 112 are biased together using spring 130, which may be made from any suitable thermoplastic or metal. Clamp 100 operates in essentially the same manner as clamp 10, described above.

This description is given for purposes of illustration and explanation. It will be apparent to those skilled in the relevant art that changes and modifications may be made to the invention described above without departing from its scope or spirit.

We claim:

1. A wound clamp, comprising:
a) a body having a first half and a second half;
b) an elastomeric band biasing the first half against the second half; and
c) a plurality of prongs associated with both the first half and the second half.

2. The wound clamp of claim 1 wherein the first half and the second half each have a groove and the elastomeric band fits within the groove.

3. The wound clamp of claim 1 wherein first half and the second half each contain cut out portions that form an aperture when the first half and the second half are biased together by the elastomeric band.

4. The wound clamp of claim 1 wherein the body comprises a lubricious material.

5. The wound clamp of claim 1 wherein the body comprises a luminous material.

6. The wound clamp of claim 3 wherein the cut out portions are flared.

7. The wound clamp of claim 3 wherein the cut out portions are beveled.

8. The wound clamp of claim 3 wherein the cut out portions are chamfered.

9. The wound clamp of claim 1 wherein the first half and the second half each contain a recess.

10. A wound clamp, comprising:
a) a body having a first half and a second half, each of the first half and the second half containing a groove and a cut out portion;
b) an elastomeric band fitting within the grooves and biasing the first half against the second half; and
c) a plurality of prongs associated with both the first half and the second half.

11. A wound clamp, comprising:
a) a body having a first half and a second half;
b) a spring biasing the first half against the second half; and
c) a plurality of prongs associated with both the first half and the second half.

12. The wound clamp of claim 11 wherein first half and the second half each contain cut out portions that form an aperture when the first half and the second half are biased together by the spring.

13. The wound clamp of claim 11 wherein the body comprises a lubricious material.

14. The wound clamp of claim 11 wherein the body comprises a luminous material.
